(19) United States
(12) Patent Application Publication
(10) Pub. No.: US 2026/0165625 A1
(43) Pub. Date: Jun. 18, 2026

(54) INTERNAL STATE ESTIMATION DEVICE AND INTERNAL STATE ESTIMATION METHOD

(71) Applicant: Mitsubishi Electric Corporation, Tokyo (JP)

(72) Inventors: Jin KATO, Tokyo (JP); Yudai NAKAMURA, Tokyo (JP); Atsushi MATSUMOTO, Tokyo (JP); Ryohei MURACHI, Tokyo (JP); Yuya OHAGI, Tokyo (JP)

(73) Assignee: Mitsubishi Electric Corporation, Tokyo (JP)

(21) Appl. No.: 19/123,829

(22) PCT Filed: Dec. 6, 2022
(86) PCT No.: PCT/JP2022/044804

§ 371 Date: Apr. 24, 2025

(51) Int. Cl. A61B 5/18; A61B 5/00; A61B 5/024; A61B 5/16

(52) U.S. Cl. CPC A61B 5/18; A61B 5/024; A61B 5/165; A61B 5/7278

(57) ABSTRACT

An internal state estimation device includes: a first extraction unit to extract a first timing at which a characteristic change occurs in biometric information on a basis of the biometric information acquired in time series from an occupant of a vehicle; a second extraction unit to extract a second timing at which a change occurs in environmental information on a basis of the environmental information which is information acquired in time series in which a change in the information affects an internal state of the occupant; a determination unit to determine an order of the first timing and the second timing; and an estimation unit to estimate an internal state of the occupant on a basis of at least one of the biometric information and the environmental information, wherein the estimation unit determines, depending on the determination result, whether or not the internal state can be estimated.

### TECHNICAL FIELD

The present disclosure relates to an internal state estimation device and an internal state estimation method.

### BACKGROUND ART

Conventionally, a technique for estimating an internal state such as an emotion of an occupant of a vehicle using biometric information of the occupant is known. For example, Patent Literature 1 discloses a technique of measuring two types of biological fluctuations, that is, heartbeat fluctuation and chaotic fluctuation, from a pulse wave of a driver, and estimating, on the basis of the two types of biological fluctuations, four states that interfere with driving of the driver, that is, an “adaptive power decrease or depressed state”, a “drowsiness or fatigue state”, a “tension or mood elevation state”, and a “stress state”.

### CITATION LIST

### Patent Literature

Patent Literature 1: JP 2020-74805 A

### SUMMARY OF INVENTION

### Technical Problem

In the technique (hereinafter, also simply referred to as “prior art”) described in Patent Literature 1, an internal state such as driver's emotion is estimated by using a pulse wave as biometric information of the driver. However, the biometric information including the pulse wave does not necessarily change in association with a change in the internal state of the driver, and may also change by an operation that does not involve a change in the internal state of the driver, such as breathing and conversation. In this case, in the above-described prior art, there is a possibility that the internal state of the driver is erroneously estimated.

The present disclosure has been made to solve the above problems, and an object of the present disclosure is to provide an internal state estimation device capable of suppressing erroneous estimation of an internal state of an occupant of a vehicle.

### Solution To Problem

An internal state estimation device according to the present disclosure includes: a first extraction unit to extract a first timing that is a timing at which a characteristic change occurs in biometric information on a basis of the biometric information acquired in time series from an occupant of a vehicle; a second extraction unit to extract a second timing, which is a timing at which a change occurs in environmental information, on a basis of the environmental information which is information acquired in time series and is information in which a change in the information affects an internal state of the occupant; a determination unit to perform determination of an order of the first timing extracted by the first extraction unit and the second timing extracted by the second extraction unit; and an estimation unit to estimate an internal state of the occupant on a basis of at least one of the biometric information and the environmental information, wherein the estimation unit determines, depending on a result of the determination performed by the determination unit, whether or not the internal state can be estimated.

### Advantageous Effects of Invention

According to the present disclosure, with the above configuration, it is possible to suppress erroneous estimation of the internal state of the occupant of the vehicle.

### BRIEF DESCRIPTION OF DRAWINGS

FIG. 1 is a diagram illustrating a configuration example of an internal state estimation device according to a first embodiment.

FIG. 2 is a flowchart illustrating an operation example of the internal state estimation device according to the first embodiment.

FIG. 3 is a diagram illustrating a specific example of biometric information (heart rate) and environmental information (gear position) in the first embodiment.

FIG. 4A is a diagram illustrating a configuration example of a first database according to the first embodiment, FIG. 4B is a diagram illustrating a configuration example of a second database according to the first embodiment, and FIG. 4C is a diagram illustrating a configuration example of a third database according to the first embodiment.

FIG. 5 is a diagram for explaining an effect of the internal state estimation device according to the first embodiment.

FIGS. 6A and 6B are diagrams illustrating a hardware configuration example of the internal state estimation device according to the first embodiment.

FIG. 7 is a diagram illustrating a configuration example of an internal state estimation device according to a second embodiment.

FIG. 8 is a diagram for explaining an example of movement of an estimation target section in the second embodiment.

FIG. 9 is a diagram illustrating a specific example of biometric information (heart rate) and environmental information (gear position) in the second embodiment.

FIG. 10 is a diagram illustrating a configuration example of an internal state estimation device according to a third embodiment.

FIG. 11A is a diagram illustrating an example of an order of a first timing and a second timing in the first embodiment, and FIG. 11B is a diagram illustrating an example of an order of the first timing and the second timing in the third embodiment.

### DESCRIPTION OF EMBODIMENTS

Hereinafter, embodiments will be described in detail with reference to the drawings.

### First Embodiment

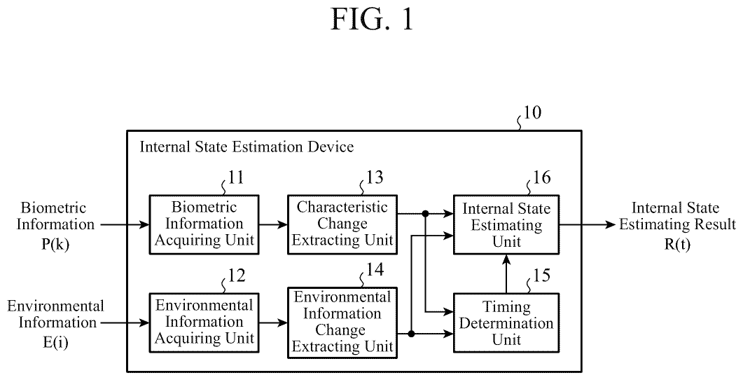

FIG. 1 is a diagram illustrating a configuration example of an internal state estimation device 10 according to a first embodiment. The internal state estimation device 10 estimates an internal state of an occupant of a vehicle. The occupant of the vehicle may be a driver of the vehicle or a fellow passenger.

For example, as illustrated in FIG. 1, the internal state estimation device 10 includes a biometric information acquiring unit 11, an environmental information acquiring unit 12, a characteristic change extracting unit (first extraction unit) 13, an environmental information change extracting unit (second extraction unit) 14, a timing determination unit (determination unit) 15, and an internal state estimating unit (estimation unit) 16.

The biometric information acquiring unit 11 is connected to a biometric information detecting unit (not illustrated) that detects biometric information P(k) from an occupant of the vehicle. The biometric information acquiring unit 11 acquires the biometric information P(k) detected in time series by the biometric information detecting unit from the biometric information detecting unit. The biometric information acquiring unit 11 outputs the acquired biometric information P(k) to the characteristic change extracting unit 13.

Here, the biometric information P(k) is, for example, at least one of information indicating a heartbeat of the occupant, information indicating a pulse wave, a pulse rate, and information indicating a face image. Furthermore, the biometric information detecting unit that detects the biometric information P(k) includes, for example, a heart rate sensor, a pulse wave sensor, a pulse rate sensor, a camera, and the like.

The environmental information acquiring unit 12 is connected to an environmental information detecting unit (not illustrated) that detects the environmental information E(i). The environmental information acquiring unit 12 acquires the environmental information E(i) detected in time series by the environmental information detecting unit from the environmental information detecting unit. The environmental information acquiring unit 12 outputs the acquired environmental information E(i) to the environmental information change extracting unit 14.

Here, the environmental information E(i) is information that affects the internal state of the occupant when the information changes, more specifically, information that causes a change in the internal state of the occupant when the information changes, and is mainly information regarding external factors other than the occupant.

Examples of the environmental information E(i) include vehicle internal information (vehicle interior information), vehicle external information (vehicle exterior information), and vehicle control information. In addition, the control information of the vehicle includes, for example, information regarding a gear position, navigation information, information regarding a steering wheel operation, information regarding a pedal operation such as an accelerator pedal and a brake pedal, and the like. The environmental information detecting unit that detects the environmental information E(i) includes, for example, a control device, a navigation system, a sensor, a camera, and the like mounted on the vehicle. Furthermore, the internal state of the occupant includes, for example, a stress state, a fatigue state, a depressed state, or the like of the occupant in addition to the emotion of the occupant.

Note that “k” in the biometric information P(k) is an input number assigned to the biometric information, and “i” in the environmental information E(i) is an input number assigned to the environmental information, and both are integers equal to or more than 1. Note that, when the input numbers k and i are viewed at any time, the values of k and i are not necessarily the same value. For example, 1 second after the internal state estimation device 10 starts the internal state estimation processing, k may be “30” and i may be “15”. This corresponds to that 30 pieces of biometric information P(k) and 15 pieces of environmental information E(i) are acquired by the internal state estimation device 10 at a time point of 1 second after the internal state estimation device 10 starts the internal state estimation processing.

The characteristic change extracting unit 13 acquires the biometric information P(k) from the biometric information acquiring unit 11. The characteristic change extracting unit 13 extracts a first timing kF, which is a timing at which a characteristic change occurs in the biometric information P(k), on the basis of the biometric information P(k) acquired from the biometric information acquiring unit 11.

For example, when a specific temporal change occurs in the biometric information P(k) acquired from the biometric information acquiring unit 11, the characteristic change extracting unit 13 determines that a characteristic change occurs in the biometric information P(k). Here, the specific temporal change of the biometric information P(k) refers to a change mainly caused by a change in the internal state of the occupant among changes of the biometric information P(k), or a change that may be caused by a change in the internal state of the occupant among changes of the biometric information P(k). For example, when the biometric information P(k) is the heart rate, the specific temporal change of the heart rate means that the heart rate acquired in time series exceeds a specific upper limit threshold, or that the heart rate is below a specific lower limit threshold.

The characteristic change extracting unit 13 outputs the extracted first timing kF to the timing determination unit 15. In addition, the characteristic change extracting unit 13 sets the biometric information P(k) used for extraction of the first timing kF as a characteristic change extracting result F(kF), and outputs the characteristic change extraction result F(kF) to the internal state estimating unit 16.

The environmental information change extracting unit 14 acquires the environmental information E(i) from the environmental information acquiring unit 12. On the basis of the environmental information E(i) acquired from the environmental information acquiring unit 12, the environmental information change extracting unit 14 extracts a second timing iZ which is a timing at which a change occurs in the environmental information E(i).

For example, when a specific temporal change occurs in the environmental information E(i) acquired from the environmental information acquiring unit 12, the environmental information change extracting unit 14 determines that a change has occurred in the environmental information E(i). The specific temporal change of the environmental information E(i) refers to, for example, when the environmental information E(i) is a gear position, a change of the gear position from a drive gear to a reverse gear, and when the environmental information E(i) is navigation information, a change of a traveling scene of a vehicle (for example, a vehicle entering a freeway from a general road).

The environmental information change extracting unit 14 outputs the extracted second timing iZ to the timing determination unit 15. Further, the environmental information change extracting unit 14 sets the environmental information E(i) used for extracting the second timing iZ as an environmental information change extracting result Z (iZ), and outputs the environmental information change extracting result Z (iZ) to the internal state estimating unit 16.

The timing determination unit 15 acquires the first timing kF output from the characteristic change extracting unit 13 and the second timing iZ output from the environmental information change extracting unit 14. Then, the timing determination unit 15 determines the order of the acquired first timing kF and second timing iZ, and outputs the determination result RT(kF, iZ) to the internal state estimating unit 16.

The internal state estimating unit 16 acquires the characteristic change extracting result F(kF) output from the characteristic change extracting unit 13 and the environmental information change extracting result Z(iZ) output from the environmental information change extracting unit 14. Furthermore, the internal state estimating unit 16 acquires the determination result RT(kF, iZ) output from the timing determination unit 15.

The internal state estimating unit 16 estimates the internal state of the occupant on the basis of at least one of the characteristic change extracting result F(kF) (that is, the biometric information P(k)) acquired from the characteristic change extracting unit 13 and the environmental information change extracting result Z (iZ) (that is, the environmental information E(i)) acquired from the environmental information change extracting unit 14. At this time, the internal state estimating unit 16 determines whether or not to be able to execute estimation of the internal state of the occupant depending on the determination result RT(kF, iZ) acquired from the timing determination unit 15.

Specifically, the internal state estimating unit 16 enables the execution of the estimation of the internal state when the timing determination unit 15 determines that the first timing kF is earlier than the second timing iZ, or when the first timing kF and the second timing iZ are determined to be the same. That is, in any of the above cases, the internal state estimating unit 16 estimates the internal state of the occupant on the basis of at least one of the characteristic change extracting result F(kF) (that is, the biometric information P(k)) acquired from the characteristic change extracting unit 13 and the environmental information change extracting result Z (iZ) (that is, the environmental information E(i)) acquired from the environmental information change extracting unit 14. Then, the internal state estimating unit 16 outputs the estimation result R(t).

On the other hand, when the timing determination unit 15 determines that the first timing kF is later than the second timing iZ, the internal state estimating unit 16 does not execute the estimation of the internal state. That is, when the timing determination unit 15 determines that the first timing kF is later than the second timing iZ, the internal state estimating unit 16 does not estimate the internal state of the occupant of the vehicle.

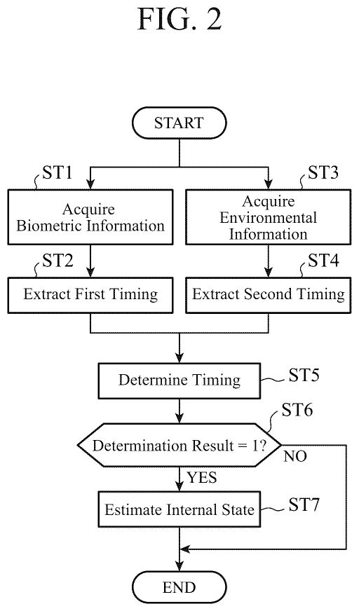

Next, an operation example of the internal state estimation device 10 according to the first embodiment will be described with reference to a flowchart illustrated in FIG. 2.

First, the biometric information acquiring unit 11 acquires biometric information P(k) detected by a biometric information detecting unit (not illustrated) in time series from the biometric information detecting unit (step ST1). The biometric information acquiring unit 11 outputs the acquired biometric information P(k) to the characteristic change extracting unit 13.

Next, the characteristic change extracting unit 13 extracts a first timing kF, which is a timing at which a characteristic change occurs in the biometric information P(k), on the basis of the biometric information P(k) acquired from the biometric information acquiring unit 11 (step ST2). In addition, the characteristic change extracting unit 13 outputs the extracted first timing kF to the timing determination unit 15, and outputs the biometric information P(k) used for extracting the first timing kF to the internal state estimating unit 16 as a characteristic change extracting result F(kF).

On the other hand, the environmental information acquiring unit 12 acquires the environmental information E(i) detected in time series by the environmental information detecting unit (not illustrated) from the environmental information detecting unit (step ST3). The environmental information acquiring unit 12 outputs the acquired environmental information E(i) to the environmental information change extracting unit 14.

Next, on the basis of the environmental information E(i) acquired from the environmental information acquiring unit 12, the environmental information change extracting unit 14 extracts a second timing iZ which is a timing at which a change occurs in the environmental information E(i) (step ST4). Further, the environmental information change extracting unit 14 outputs the extracted second timing iZ to the timing determination unit 15, and outputs the environmental information E(i) used for extracting the second timing iZ to the internal state estimating unit 16 as an environmental information change extracting result Z(iZ).

Note that the steps ST1 to ST2 and steps ST3 to ST4 described above may be performed in parallel.

Next, the timing determination unit 15 determines the order of the first timing kF and the second timing iZ (step ST5). Furthermore, the timing determination unit 15 outputs the determination result RT(kF, iZ) to the internal state estimating unit 16.

For example, the timing determination unit 15, when determining that the first timing kF is earlier than the second timing iZ or the first timing kF and the second timing iZ are the same as a result of the determination, sets the determination result RT(kF, iZ) to “1”. On the other hand, when determining that the first timing kF is later than the second timing iZ as a result of the determination, the timing determination unit 15 sets the determination result RT(kF, iZ) to “0”.

Next, the internal state estimating unit 16 checks whether the determination result RT(kF, iZ) acquired from the timing determination unit 15 is “1”, that is, whether the first timing kF is earlier than the second timing iZ, or whether or not the first timing kF and the second timing iZ are determined to be the same (step ST6). As a result, if the determination result RT(kF, iZ) is not “1” (step ST6; NO), the internal state estimating unit 16 determines not to execute the estimation of the internal state, and ends the processing. On the other hand, if the determination result RT(kF, iZ) is “1” (step ST6; YES), the process proceeds to step ST7.

In step ST7, the internal state estimating unit 16 enables execution of estimation of the internal state, and estimates the internal state of the occupant of the vehicle on the basis of at least one of the characteristic change extracting result F(kF) (that is, the biometric information P(k)) acquired from the characteristic change extracting unit 13 and the environmental information change extracting result Z(iZ) (that is, the environmental information E(i)) acquired from the environmental information change extracting unit 14 (step ST7). Then, the internal state estimating unit 16 outputs the estimation result R(t).

Next, an operation example of the internal state estimation device 10 including an internal state estimation method by the internal state estimating unit 16 will be described with a specific example.

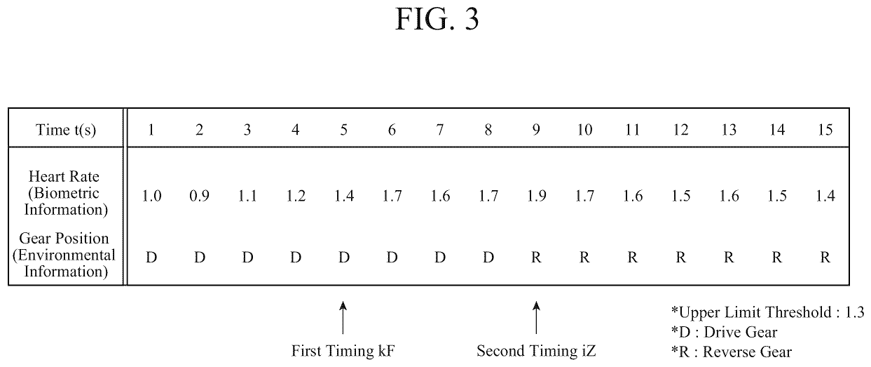

Here, in order to make the description easy to understand, it is assumed that the biometric information P(k) is the heart rate of the occupant and the environmental information E(i) is the gear position of the vehicle. In addition, in the following description, it is assumed that the biometric information acquiring unit 11 acquires a total of 15 heart rates of the occupant one by one in 1 second, and the environmental information acquiring unit 12 acquires a total of 15 gear positions of the vehicle one by one in 1 second, starting from the time when the internal state estimation device 10 starts the operation. FIG. 3 illustrates a specific example of the heart rate and the gear position in the above case.

In addition, here, the specific temporal change of the heart rate, which is a reference for determining that a characteristic change has occurred in the heart rate, means that the heart rate has exceeded “1.3” which is the upper limit threshold.

First, the characteristic change extracting unit 13 extracts “5 seconds (t=5)”, which is the timing at which the heart rate exceeds the upper limit threshold of 1.3, as the first timing kF (corresponding to step ST2 described above), and outputs the first timing kF to the timing determination unit 15. In addition, the characteristic change extracting unit 13 outputs 15 heart rates (15 heart rates from “1.0” at t=1 to “1.4” at t=15) used for extracting the first timing kF to the internal state estimating unit 16 as characteristic change extracting results F(kF).

On the other hand, the environmental information change extracting unit 14 extracts “9 seconds (t=9)”, which is the timing at which the gear position changes from the drive gear (indicated by D in FIG. 3) to the reverse gear (indicated by R in FIG. 3), as the second timing iZ (corresponding to step ST4 described above), and outputs the second timing iZ to the timing determination unit 15. Further, the environmental information change extracting unit 14 outputs 15 gear positions (15 gear positions from “D” at t=1 to “R” at t=15) used for extracting the second timing iZ to the internal state estimating unit 16 as the environmental information change extracting result Z(iZ).

Next, the timing determination unit 15 determines the order of “5 seconds (t=5)” that is the first timing kF and “9 seconds (t=9)” that is the second timing iZ, and determines that the first timing kF is earlier (corresponding to step ST5 described above). In addition, since determining that the first timing kF is earlier, the timing determination unit 15 sets the determination result RT(kF, iZ) to “1”, and outputs the determination result RT(kF, iZ) to the internal state estimating unit 16.

Next, the internal state estimating unit 16 checks whether or not the determination result RT(kF, iZ) acquired from the timing determination unit 15 is “1” (corresponding to step ST6 described above). As a result, since the determination result RT(kF, iZ) is “1”, the internal state estimating unit 16 enables execution of estimation of the internal state, and estimates the internal state of the occupant on the basis of at least one of the 15 heart rates and the 15 gear positions (corresponding to step ST7 described above).

Here, an example of an internal state estimation method by the internal state estimating unit 16 will be described. The internal state estimating unit 16 estimates the internal state of the occupant using, for example, a database for internal state estimation generated in advance. Note that this database is recorded in advance in a recording unit (not illustrated) included in the internal state estimation device 10.

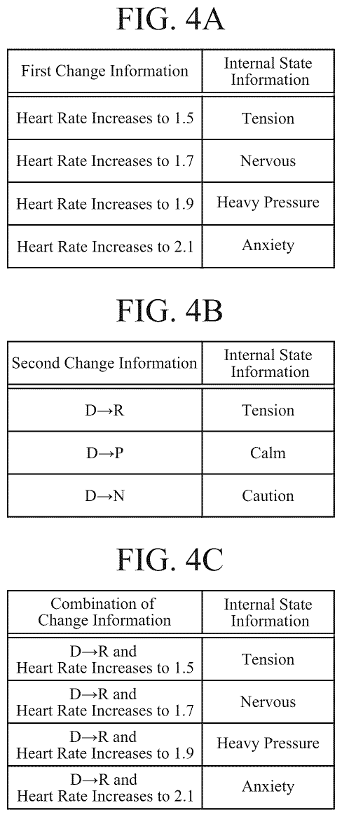

An example of the database is illustrated in FIG. 4A. As illustrated in FIG. 4A, the database (hereinafter, also referred to as a “first database”) is configured by associating information (hereinafter, also referred to as “first change information”) defining a mode of change (transition) of 15 pieces of biometric information P(k) derived from the characteristic change extracting result F(kF) with information (hereinafter, also referred to as “internal state information”) indicating an internal state of the occupant.

For example, in the example of FIG. 4A, “the heart rate has increased (exceeding 1.3 which is the upper limit threshold) to 1.5” is set as the first change information, and “tension” is set as the internal state information associated with the first change information. Similarly, in the example of FIG. 4A, “the heart rate has increased (exceeding 1.3 which is the upper limit threshold) to 1.7” is set as the first change information, and “nervous” is set as the internal state information associated with the first change information. Note that, although not explicitly illustrated in FIG. 4A, the change in the biometric information P(k) indicated by the first change information also includes the characteristic change (exceeding 1.3 which is the upper limit threshold) occurring at the above-described first timing kF.

Similarly, in the example of FIG. 4A, “the heart rate has increased (exceeding 1.3 which is the upper limit threshold) to 1.9” is set as the first change information, and “heavy pressure” is set as the internal state information associated with the first change information. Furthermore, in the example of FIG. 4A, “the heart rate has increased (exceeding 1.3 which is the upper limit threshold) to 2.1” is set as the first change information, and “anxiety” is set as the internal state information associated with the first change information.

For example, in the example of FIG. 3, it can be seen that the heart rate has increased to 1.9 exceeding 1.3 which is the upper limit threshold by 15 heart rates which are the characteristic change extracting results F(kF). Therefore, the internal state estimating unit 16 searches the first database with the fact that the heart rate has increased to 1.9 (that is, a mode of change in heart rate) as a key, and specifies “heavy pressure” which is the internal state information corresponding to the key. Then, the internal state estimating unit 16 outputs an estimation result of the internal state of the occupant as “heavy pressure” on the basis of the specified internal state information.

Note that, here, an operation example in a case where the heart rate increases exceeding 1.3 which is the upper limit threshold has been described. However, the same applies to a case where the heart rate falls below the lower limit threshold (for example, 0.7). In this case, in the first database, for example, “the heart rate has decreased to 0.6 (below 0.7 that is the lower limit threshold)” may be set as the first change information, and for example, “fatigue” may be set as the internal state information associated with the first change information.

Next, another example of the database is illustrated in FIG. 4B. As illustrated in FIG. 4B, the database (hereinafter, also referred to as a “second database”) is configured by associating information (hereinafter, also referred to as “second change information”) defining a mode of change (transition) of the environmental information E(i) derived from the environmental information change extracting result Z(iZ) with information (internal state information) indicating the internal state of the occupant.

Note that the change in the environmental information E(i) indicated by the second change information also includes the change in the environmental information E(i) occurring at the second timing iZ described above.

For example, in the example of FIG. 4B, “the gear position has been changed from the drive gear to the reverse gear” is set as the second change information, and “tension” is set as the internal state information associated with the second change information. In the example of FIG. 4B, “the gear position has been changed from the drive gear to the parking gear (P)” is set as the second change information, and “calm” is set as the internal state information associated with the second change information.

In the example of FIG. 4B, “the gear position has been changed from the drive gear to the neutral gear (N)” is set as the second change information, and “caution” is set as the internal state information associated with the second change information.

For example, in the example of FIG. 3, it can be seen that the gear position has been changed from the drive gear to the reverse gear by 15 gear positions that are the environmental information change extracting result Z(iZ). Therefore, the internal state estimating unit 16 searches the second database with the fact that the gear position has been changed from the drive gear to the reverse gear (that is, a mode of change in gear position) as a key, and specifies “tension” which is the internal state information corresponding to the key. Then, the internal state estimating unit 16 outputs an estimation result of the internal state of the occupant as “tension” on the basis of the specified internal state information.

Note that, although the operation example in a case where the environmental information E(i) indicates the gear position has been described here, the same applies to a case where the environmental information E(i) indicates other than the gear position. For example, if the environmental information E(i) is operation information of a brake pedal, for example, “the fact that the number of times of depression of the brake pedal during the predetermined time increased to 8 (exceeding 5 which is the upper limit threshold)” may be set in the second database as the second change information, and for example, “impatient” may be set as the internal state information associated with the second change information.

Next, another example of the database is illustrated in FIG. 4C. As illustrated in FIG. 4C, this database (hereinafter, also referred to as a “third database”) is formally configured by combining the first database and the second database described above. Specifically, the third database is configured by associating the combination of the first change information and the second change information with the internal state information.

For example, in the example of FIG. 4C, the fact that “the gear position has been changed from drive gear to reverse gear and the heart rate increased to 1.5 (exceeding 1.3 which is the upper limit threshold)” is set as the combination of the change information, and “tension” is set as the internal state information associated with the combination. Furthermore, for example, in the example of FIG. 4C, the fact that “the gear position has been changed from drive gear to reverse gear and the heart rate increased to 1.7 (exceeding 1.3 which is the upper limit threshold)” is set as the combination of the change information, and “nervous” is set as the internal state information associated with the combination.

Similarly, in the example of FIG. 4C, the fact that “the gear position has been changed from drive gear to reverse gear and the heart rate increased to 1.9 (exceeding 1.3 which is the upper limit threshold)” is set as the combination of the change information, and “heavy pressure” is set as the internal state information associated with the combination. Furthermore, in the example of FIG. 4C, the fact that “the gear position has been changed from drive gear to reverse gear and the heart rate increased to 2.1 (exceeding 1.3 which is the upper limit threshold)” is set as the combination of the change information, and “anxiety” is set as the internal state information associated with the combination.

For example, in the example of FIG. 3, it can be seen that the heart rate has increased to 1.9 exceeding 1.3 which is the upper limit threshold by 15 heart rates which are the characteristic change extracting results F(kF) and 15 gear positions which are the environmental information change extracting result Z(iZ), and the gear position has been changed from the drive gear to the reverse gear. Therefore, the internal state estimating unit 16 searches the third database with the fact that the gear position has been changed from the drive gear to the reverse gear and the heart rate has increased to 1.9 (that is, modes of changes in both the gear position and the heart rate) as a key, and specifies “heavy pressure” which is the internal state information corresponding to the key. Then, the internal state estimating unit 16 outputs an estimation result of the internal state of the occupant of the vehicle as “heavy pressure” on the basis of the specified internal state information.

The internal state estimating unit 16 can accurately estimate the internal state of the occupant by estimating the internal state of the occupant using, for example, any one of the first database to the third database. Note that, if the internal state estimating unit 16 estimates the internal state of the occupant using the third database, the internal state estimating unit can estimate the internal state of the occupant on the basis of the relationship between the heart rate that is the biometric information P(k) and the gear position that is the environmental information E(i). Therefore, the estimation accuracy can be improved as compared with the case of estimating the internal state of the occupant using the first database or the second database.

Note that, in a case where the emotion of the occupant of the vehicle is used as the internal state indicated by the internal state information set in each of the above-described databases, for example, an emotion model defined in Russell's circumplex model of emotion can be used for construction of each of the databases.

Russell's circumplex model of emotion is a model indicating that all emotions are arranged in a circular shape on a plane represented two-dimensionally by “pleasant unpleasant” and “awakening-non-awakening”. Various emotion models have been proposed, but Russell's circumplex model of emotion is expressed in a simple structure and can be applied exhaustively for all emotions, and thus is suitably used for constructing each of the above-described databases.

For example, in Russell's circumplex model of emotion, “tension”, “nervous”. “heavy pressure”, “anxiety”, and the like are defined as emotions classified into “unpleasant” and “awakening”, and “sadness”, “depression”, “lethargy”, “fatigue”, and the like are defined as emotions classified into “unpleasant” and “non-awakening”.

Furthermore, in the same model, “caution”, “excitement”, “energetic”, “happy”, and the like are defined as emotions classified into “pleasant” and “awakening”, and “satisfaction”, “pleasant”, “relaxing”, “calm”, and the like are defined as emotions classified into “pleasant” and “non-awakening”.

The administrator or the like of the internal state estimation device 10 can construct each of the above-described databases, for example, by using each of the above-described emotions as an internal state. As illustrated in FIGS. 4A to 4C, at least one of the first change information and the second change information needs to be associated with each internal state.

Note that the contents of the first change information, the second change information, and the internal state information in each database described above are merely examples, and contents other than the above may be set. For example, in the above description, the example of using the emotion (tension or the like) of the occupant as the internal state information has been described, but the internal state information is not limited thereto, and for example, a stress state, a fatigue state, a depression state, or the like of the occupant may be used. Furthermore, in the above example, an example of setting the name (“tension ” or the like) of the emotion of the occupant as the internal state information in each database has been described. However, the internal state information in each database is not limited thereto, and for example, information obtained by digitizing the emotion of the occupant may be set.

Further, in each database described above, for example, different information may be set for each occupant. For example, the occupant may appropriately select the emotion (for example, impatient, tension, energetic, and the like) that the occupant desires to estimate as the internal state of the occupant and the type (for example, gear position, navigation information, and the like) of the environmental information E(i), and generate the databases in advance on the basis of the selected emotion and a type of the environmental information E(i). In this case, the occupant can use a database customized for estimating an internal state of the occupant, and convenience is improved.

Furthermore, in the above description, an example has been described in which the internal state estimating unit 16 uses the determination result RT(kF, iZ) of the order of the first timing kF and the second timing iZ acquired from the timing determination unit 15 when determining whether or not to perform the internal state estimation processing. However, the internal state estimating unit 16 is not limited thereto, and for example, the determination result RT(kF, iZ) acquired from the timing determination unit 15 may be used as it is for estimating the internal state of the occupant.

For example, if the determination result RT(kF, iZ) acquired from the timing determination unit 15 is “1”, that is, if the first timing kF is earlier than the second timing iZ, or if the first timing kF and the second timing iZ are the same, the internal state estimating unit 16 may output an estimation result R(t) indicating that the internal state of the occupant is “tension”.

Furthermore, in the above description, an example in which one first timing kF and one second timing iZ are extracted has been described. However, it is also assumed that a plurality of at least one of the first timing kF and the second timing iZ are extracted. In that case, the timing determination unit 15 may set the determination result RT(kF, iZ) to “1” if there is at least one case where the first timing kF comes before the second timing iZ or the first timing kF and the second timing iZ are the same in the extracted timing group.

Furthermore, the internal state estimating unit 16 may estimate the internal state of the occupant using, for example, a machine learning model generated for internal state estimation. In that case, the machine learning model may be, for example, any one of (1) a model learned so as to output internal state information corresponding to first change information in response to an input of the first change information derived from the characteristic change extracting result F(kF), (2) a model learned to output internal state information corresponding to second change information in response to an input of the second change information derived from the environmental information change extracting result Z(iZ), and (3) a model learned to output internal state information corresponding to a combination of the first change information and the second change information in response to an input of a combination of the first change information and the second change information.

Furthermore, the internal state estimating unit 16 may estimate the internal state of the occupant by a method similar to the above-described method in the prior art. For example, the internal state estimating unit 16 may measure two types of biological fluctuations of heart rate fluctuation and chaotic fluctuation from the pulse wave of the occupant acquired as the biometric information P(k), and estimate the internal state of the occupant on the basis of the two types of biological fluctuations.

Furthermore, the internal state estimating unit 16 may output the estimation result of the internal state of the occupant in two levels such as “anxiety” or “impatient”, or may output the estimation result in multiple levels (for example, 0-100 levels) such as “anxiety in 90/100 levels”.

As described above, in the internal state estimation device 10 according to the first embodiment, the internal state estimating unit 16 determines whether or not to be able to execute estimation of the internal state of the occupant depending on the determination result by the timing determination unit 15. Specifically, the internal state estimating unit 16 enables the execution of the estimation of the internal state when the timing determination unit 15 determines that the first timing kF is earlier than the second timing iZ, or when the first timing kF and the second timing iZ are determined to be the same. On the other hand, when the timing determination unit 15 determines that the first timing kF is later than the second timing iZ, the internal state estimating unit 16 does not execute the estimation of the internal state. This is based on the idea that when the internal state of the occupant changes (for example, the occupant becomes nervous), the biometric information changes (for example, the heart rate greatly increases) before the environmental information changes (for example, the gear position is changed from the drive gear to the reverse gear). Therefore, in the internal state estimation device 10 according to the first embodiment, it is possible to suppress erroneous estimation of the internal state of the occupant on the basis of a change in biometric information without a change in the internal state of the occupant.

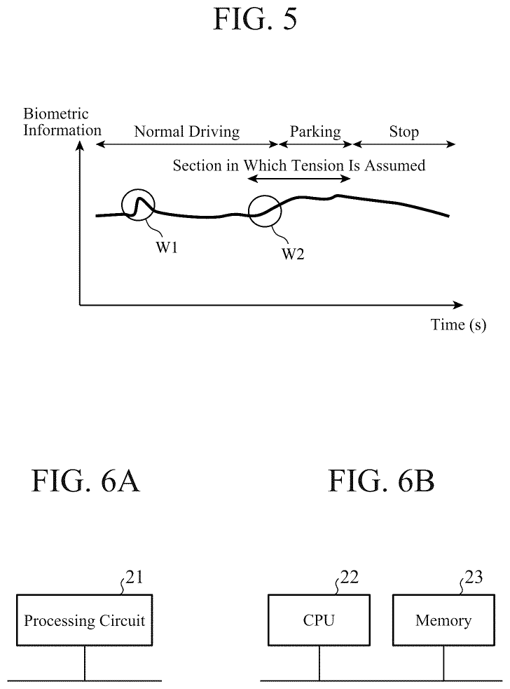

In this regard, in the prior art described above, an internal state such as driver's emotions is estimated by using a pulse wave as driver's biometric information. However, the biometric information including the pulse wave may change not only by the change in the internal state of the driver at the time of switching from the normal driving to the parking operation as illustrated in W2 of FIG. 5, but also by the operation without the change in the internal state of the driver such as breathing and conversation during the normal driving as illustrated in W1 of FIG. 5. In this case, in the above-described prior art, there is a possibility that a change in W1 is captured and the internal state of the driver is erroneously estimated as “tension” or the like. On the other hand, in the internal state estimation device 10 according to the first embodiment, the occurrence of such a trouble is suppressed.

Next, a hardware configuration example of the internal state estimation device 10 according to the first embodiment will be described with reference to FIG. 6. The functions of the biometric information acquiring unit 11, the environmental information acquiring unit 12, the characteristic change extracting unit 13, the environmental information change extracting unit 14, the timing determination unit 15, and the internal state estimating unit 16 in the internal state estimation device 10 are implemented by a processing circuit. The processing circuit may be dedicated hardware as illustrated in FIG. 6A, or may be a central processing unit (CPU, also referred to as a central processor, a processing unit, an arithmetic unit, a microprocessor, a microcomputer, a processor, or a digital signal processor (DSP))22 that executes a program stored in a memory 23 as illustrated in FIG. 6B.

In a case where the processing circuit is dedicated hardware, the processing circuit 21 corresponds to, for example, a single circuit, a composite circuit, a programmed processor, a parallel programmed processor, an application specific integrated circuit (ASIC), a field programmable gate array (FPGA), or a combination thereof. The functions of the biometric information acquiring unit 11, the environmental information acquiring unit 12, the characteristic change extracting unit 13, the environmental information change extracting unit 14, the timing determination unit 15, and the internal state estimating unit 16 may be implemented by the processing circuit 21, or the functions of the respective units may be collectively implemented by the processing circuit 21.

When the processing circuit is the CPU22, the functions of the biometric information acquiring unit 11, the environmental information acquiring unit 12, the characteristic change extracting unit 13, the environmental information change extracting unit 14, the timing determination unit 15, and the internal state estimating unit 16 are implemented by software, firmware, or a combination of software and firmware. The software and the firmware are described as programs and stored in the memory 23. The processing circuit reads and executes the program stored in the memory 23 to implement the function of each unit. That is, the internal state estimation device 10 includes a memory for storing a program that results in execution of each step illustrated in FIG. 2, for example, when executed by the processing circuit. It can also be said that these programs cause a computer to execute procedures and methods performed by the biometric information acquiring unit 11, the environmental information acquiring unit 12, the characteristic change extracting unit 13, the environmental information change extracting unit 14, the timing determination unit 15, and the internal state estimating unit 16. Here, examples of the memory 23 include a nonvolatile or volatile semiconductor memory such as a random access memory (RAM), a read only memory (ROM), a flash memory, an erasable programmable ROM (EPROM), or an electrically EPROM (EEPROM), a magnetic disk, a flexible disk, an optical disk, a compact disc, a mini disc, a digital versatile disc (DVD), or the like.

Note that some of the functions of the biometric information acquiring unit 11, the environmental information acquiring unit 12, the characteristic change extracting unit 13, the environmental information change extracting unit 14, the timing determination unit 15, and the internal state estimating unit 16 may be implemented by dedicated hardware, and others may be implemented by software or firmware. For example, the functions of the biometric information acquiring unit 11 can be implemented by a processing circuit as dedicated hardware, and the functions of the environmental information acquiring unit 12, the characteristic change extracting unit 13, the environmental information change extracting unit 14, the timing determination unit 15, and the internal state estimating unit 16 can be implemented by the processing circuit reading and executing programs stored in the memory 23.

As described above, the processing circuit can implement the above-described functions by hardware, software, firmware, or a combination thereof.

As described above, according to the first embodiment, the internal state estimation device 10 includes the characteristic change extracting unit 13 that extracts the first timing kF that is the timing at which the characteristic change occurs in the biometric information on the basis of the biometric information P(k) acquired in time series from the occupant of the vehicle, the environmental information change extracting unit 14 that extracts the second timing iZ that is the timing at which the change occurs in the environmental information on the basis of the environmental information E(i) that is the information acquired in time series and is the information in which the change in the information affects the internal state of the occupant, the timing determination unit 15 that determines the order of the first timing kF extracted by the characteristic change extracting unit 13 and the second timing iZ extracted by the environmental information change extracting unit 14, and the internal state estimating unit 16 that estimates the internal state of the occupant on the basis of at least one of the biometric information P(k) and the environmental information E(i), in which the internal state estimating unit 16 determines whether or not to be able to estimate the internal state depending on a determination result by the timing determination unit 15. As a result, the internal state estimation device 10 according to the first embodiment can suppress erroneous estimation of the internal state of the occupant.

Furthermore, the internal state estimating unit 16 enables estimation of the internal state when the timing determination unit 15 determines that the first timing kF is earlier than the second timing iZ, or when the first timing kF and the second timing iZ are determined to be the same. As a result, the internal state estimation device 10 according to the first embodiment can accurately suppress erroneous estimation of the internal state of the occupant.

Furthermore, the internal state estimating unit 16 estimates the internal state of the occupant using a database in which at least one of first change information that is information defining a mode of change in the biometric information P(k) including a characteristic change at the first timing kF and second change information that is information defining a mode of change in the environmental information E(i) including a change at the second timing iZ is associated with information indicating the internal state of the occupant. As a result, the internal state estimation device 10 according to the first embodiment can accurately estimate the internal state of the occupant.

The biometric information P(k) is at least one of information indicating the heartbeat of the occupant, information indicating the pulse rate of the occupant, and information indicating the face image of the occupant. As a result, the internal state estimation device 10 according to the first embodiment can suppress erroneous estimation of the internal state of the occupant by using information that can be easily acquired from the occupant.

The environmental information E(i) is at least one of vehicle internal information, vehicle external information, and vehicle control information. As a result, the internal state estimation device 10 according to the first embodiment can suppress erroneous estimation of the internal state of the occupant by using information that can be easily acquired from the vehicle.

### Second Embodiment

In the first embodiment, the internal state estimation device that determines whether or not to be able to estimate the internal state of the occupant depending on the determination result of the order of the first timing kF and the second timing iZ has been described. In a second embodiment, an internal state estimation device that sets an estimation target section having a predetermined time range on a time series in which biometric information P(k) and environmental information E(i) are acquired and determines whether or not to be able to estimate an internal state for each estimation target section will be described.

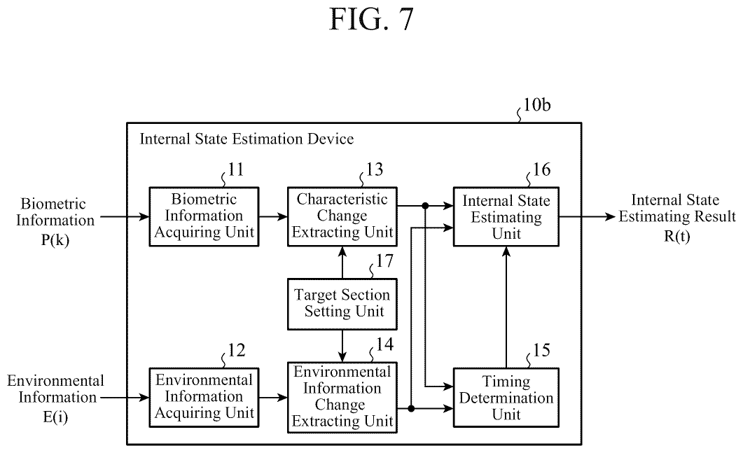

FIG. 7 is a diagram illustrating a configuration example of an internal state estimation device 10b according to the second embodiment. The internal state estimation device 10b according to the second embodiment is obtained by adding a target section setting unit 17 to the internal state estimation device 10 according to the first embodiment illustrated in FIG. 1. Since the other configurations of the internal state estimation device 10b according to the second embodiment are the same as those of the internal state estimation device 10 according to the first embodiment illustrated in FIG. 1, the same reference numerals are given and the description thereof will be omitted.

The target section setting unit 17 sets an estimation target section L having a predetermined time range on the time series in which the biometric information P(k) and the environmental information E(i) are acquired. The estimation target section L defines a section in which the internal state estimation device 10b estimates the internal state of the occupant on the time series in which the biometric information P(k) and the environmental information E(i) are acquired.

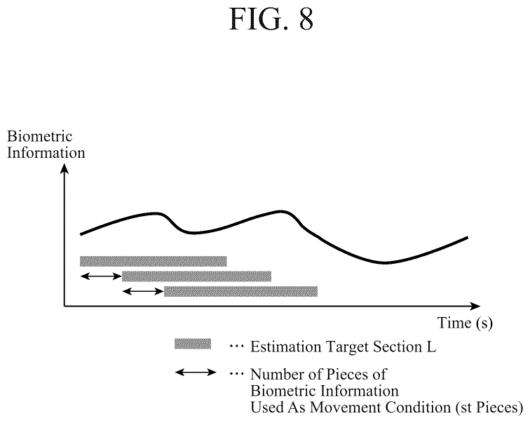

For example, as illustrated in FIG. 8, the target section setting unit 17 can move the estimation target section L in a time direction (direction in which time advances) every time a predetermined condition is satisfied. The predetermined condition is that, for example, the biometric information acquiring unit 11 acquires st pieces of biometric information P(k) (st represents an integer equal to or more than 1). In this case, the target section setting unit 17 moves the estimation target section L in the time direction every time st pieces of biometric information P(k) are acquired by the biometric information acquiring unit 11.

In the internal state estimation device 10b according to the second embodiment, when the target section setting unit 17 sets the estimation target section L, the characteristic change extracting unit 13 extracts the first timing kF, the environmental information change extracting unit 14 extracts the second timing iZ, the timing determination unit 15 performs the order determination, and the internal state estimating unit 16 performs the estimation of the internal state, within the set range of the estimation target section L. In addition, in the internal state estimation device 10b, when the target section setting unit 17 moves the estimation target section L, each processing described above is performed for each estimation target section L after the movement.

For example, the characteristic change extracting unit 13 extracts the first timing kF on the basis of Tp pieces of biometric information P(k−Tp+1) to P(k) included in the estimation target section L set by the target section setting unit 17 among the pieces of biometric information P(k) acquired in time series from the occupant. In addition, the characteristic change extracting unit 13 outputs the extracted first timing kF to the timing determination unit 15, and outputs Tp pieces of biometric information P(k−Tp+1) to P(k) used for extracting the first timing kF to the internal state estimating unit 16 as characteristic change extracting results F(kF). Note that Tp is, for example, an integer equal to or more than 2.

Further, the environmental information change extracting unit 14 extracts the second timing iZ on the basis of Tp pieces of environmental information E(i−Tp+1) to E(i) included in the estimation target section L set by the target section setting unit 17 among pieces of environmental information E(i) acquired in time series from the vehicle and the surrounding area of the vehicle. Further, the environmental information change extracting unit 14 outputs the extracted second timing iZ to the timing determination unit 15, and outputs Tp pieces of environmental information E(i−Tp+1) to E(i) used for extracting the second timing iZ to the internal state estimating unit 16 as environmental information change extracting results Z(iZ).

In addition, the timing determination unit 15 determines the order of the first timing kF extracted by the characteristic change extracting unit 13 on the basis of the Tp pieces of biometric information P(k−Tp+1) to P(k) included in the estimation target section L and the second timing iZ extracted by the environmental information change extracting unit 14 on the basis of the Tp pieces of environmental information E(i−Tp+1) to E(i) included in the estimation target section L.

When the timing determination unit 15 determines that the first timing kF is earlier than the second timing iZ or that the first timing kF and the second timing iZ are the same, the internal state estimating unit 16 enables execution of the estimation of the internal state, and estimates the internal state of the occupant of the vehicle on the basis of at least one of the characteristic change extracting results F(kF) (that is, the Tp pieces of biometric information P(k−Tp+1) to P(k)) acquired from the characteristic change extracting unit 13 and the environmental information change extracting results Z(iZ) (that is, the Tp pieces of environmental information E(i−Tp+1) to E(i)) acquired from the environmental information change extracting unit 14. On the other hand, when the timing determination unit 15 determines that the first timing kF is later than the second timing iZ, the internal state estimating unit 16 does not estimate the internal state in the estimation target section L.

Next, an operation example of the internal state estimation device 10b including an internal state estimation method by the internal state estimating unit 16 will be described with a specific example.

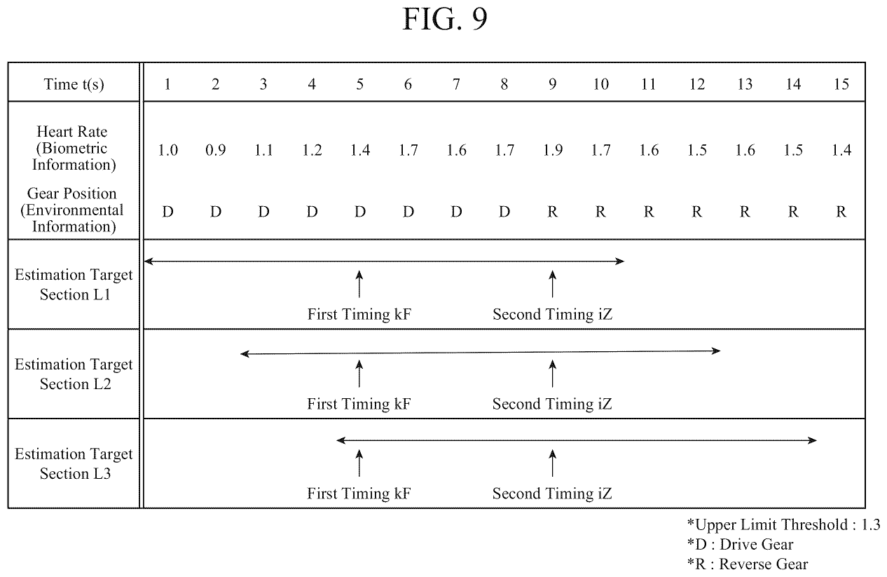

Here, in order to make the description easy to understand, a specific example similar to that of the first embodiment will be described. That is, it is assumed that the biometric information P(k) is the heart rate of the occupant, the environmental information E(i) is the gear position of the vehicle, the biometric information acquiring unit 11 acquires a total of 15 heart rates of the occupant one by one in 1 second, and the environmental information acquiring unit 12 acquires a total of 15 gear positions of the vehicle one by one in 1 second. In addition, here, the specific temporal change of the heart rate, which is a reference for determining that a characteristic change has occurred in the heart rate, means that the heart rate has exceeded “1.3” which is the upper limit threshold. A specific example of the heart rate and the gear position in the above case is illustrated in FIG. 9.

First, the target section setting unit 17 sets the estimation target section L having a predetermined time range on the time series in which the biometric information P(k) and the environmental information E(i) are acquired. Here, assuming that the predetermined time range is “10 seconds”, the target section setting unit 17 sets, for example, a section from 1 second (t=1) to 10 seconds (t=10) as the first estimation target section L1.

In this case, the characteristic change extracting unit 13 refers to the 10 pieces of biometric information P(1) to P(10) included in the estimation target section L1, extracts “5 seconds (t=5)”, which is the timing at which the heart rate exceeds 1.3 which is the upper limit threshold, as the first timing kF in the 10 pieces of biometric information, and outputs the first timing kF to the timing determination unit 15. In addition, the characteristic change extracting unit 13 outputs 10 heart rates (10 heart rates from “1.0” at t=1 to “1.7” at t=10) used for extracting the first timing kF to the internal state estimating unit 16 as characteristic change extracting results F(kF).

On the other hand, the environmental information change extracting unit 14 refers to the 10 pieces of environmental information E(1) to E(10) included in the estimation target section L1, extracts “9 seconds (t=9)”, which is the timing at which the gear position changes from the drive gear to the reverse gear in the 10 pieces of environmental information, as the second timing iZ, and outputs the second timing iZ to the timing determination unit 15. Further, the environmental information change extracting unit 14 outputs 10 gear positions (10 gear positions from “D” at t=1 to “R” at t=10) used for extracting the second timing iZ to the internal state estimating unit 16 as the environmental information change extracting result Z(iZ).

Next, the timing determination unit 15 determines the order of “5 seconds (t=5)” that is the first timing kF and “9 seconds (t=9)” that is the second timing iZ, and determines that the first timing kF is earlier. In addition, since determining that the first timing kF is earlier, the timing determination unit 15 sets the determination result RT(kF, iZ) to “1”, and outputs the determination result RT(kF, iZ) to the internal state estimating unit 16.

Next, the internal state estimating unit 16 checks whether or not the determination result RT(kF, iZ) acquired from the timing determination unit 15 is “1”. As a result, since the determination result RT(kF, iZ) is “1”, the internal state estimating unit 16 enables execution of estimation of the internal state, and estimates the internal state of the occupant in the first estimation target section L1 on the basis of at least one of the 10 heart rates and the 10 gear positions. The internal state estimation method by the internal state estimating unit 16 is similar to the method described in the first embodiment.

Here, the target section setting unit 17 is movable in the estimation target section L in the time direction every time a predetermined condition is satisfied, and the predetermined condition is that, for example, two pieces of biometric information P(k) are acquired by the biometric information acquiring unit 11. In this case, the target section setting unit 17 moves the estimation target section L1 in the time direction at a time point of 2 seconds (t=2), which is the timing at which two pieces of biometric information P(k) are acquired by the biometric information acquiring unit 11, and sets the next estimation target section L2. The next estimation target section L2 is 10 seconds from 3 seconds (t=3) to 12 seconds (t=12).

When the next estimation target section L2 is set, the characteristic change extracting unit 13, the environmental information change extracting unit 14, the timing determination unit 15, and the internal state estimating unit 16 perform the above-described processing on the basis of the 10 pieces of biometric information and environmental information included in the estimation target section L2. At this time, the internal state of the occupant estimated by the internal state estimating unit 16 becomes the internal state of the occupant in the estimation target section L2.

Thereafter, the target section setting unit 17 moves the estimation target section L2 in the time direction at a time point of 4 seconds (t=4), which is the timing at which two pieces of biometric information P(k) are acquired by the biometric information acquiring unit 11 next, and sets the next estimation target section L3. The next estimation target section L3 is 10 seconds from 5 seconds (t=5) to 14 seconds (t=14).

When the next estimation target section L3 is set, the characteristic change extracting unit 13, the environmental information change extracting unit 14, the timing determination unit 15, and the internal state estimating unit 16 perform the above-described processing on the basis of the 10 pieces of biometric information and environmental information included in the estimation target section L3. At this time, the internal state of the occupant estimated by the internal state estimating unit 16 becomes the internal state of the occupant in the estimation target section L3.

The internal state estimation device 10b according to the second embodiment estimates the internal state of the occupant for each moved estimation target section L while the target section setting unit 17 moves the estimation target section L in the time direction in a similar manner.

As described above, by including the target section setting unit 17, the internal state estimation device 10b according to the second embodiment can obtain an estimation result of the internal state of the occupant for a desired section on a time series. Furthermore, the internal state estimation device 10b according to the second embodiment can continuously estimate the internal state of the occupant in more detail by moving the estimation target section L in the time direction.

As described above, according to the second embodiment, the internal state estimation device 10b includes the target section setting unit 17 that sets the estimation target section L having the predetermined time range on the time series in which the biometric information P(k) and the environmental information E(i) are acquired, the characteristic change extracting unit 13 extracts the first timing kF on the basis of the biometric information included in the estimation target section L set by the target section setting unit 17 among the pieces of biometric information acquired in time series from the occupant, the environmental information change extracting unit 14 extracts the second timing iZ on the basis of the environmental information included in the estimation target section L set by the target section setting unit 17 among the pieces of environmental information acquired in time series, the timing determination unit 15 determines the order of the first timing kF extracted by the characteristic change extracting unit 13 on the basis of the biometric information included in the estimation target section L and the second timing iZ extracted by the environmental information change extracting unit 14 on the basis of the environmental information included in the estimation target section L, and the internal state estimating unit 16 estimates the internal state of the occupant on the basis of at least one of the biometric information and the environmental information included in the estimation target section L, and determines whether or not to be able to estimate the internal state depending on the determination result by the timing determination unit 15. As a result, the internal state estimation device 10b according to the second embodiment can obtain an estimation result of the internal state of the occupant for a desired section on a time series in addition to the effects of the first embodiment.

The target section setting unit 17 moves the estimation target section L in the time direction every time a predetermined condition is satisfied. As a result, the internal state estimation device 10b according to the second embodiment can continuously estimate the internal state of the occupant in more detail.

In addition, the predetermined condition is that st pieces of biometric information P(k) (st represents an integer equal to or more than 1) are acquired. As a result, the internal state estimation device 10b according to the second embodiment can move the estimation target section L in dependence on the acquired number of pieces of biometric information.

### Third Embodiment

In the first embodiment, the internal state estimation device that determines whether or not to be able to execute the processing of estimating the internal state of the occupant depending on the determination result of the order of the first timing and the second timing has been described. In a third embodiment, an internal state estimation device capable of more precisely determining an order of a first timing and a second timing will be described.

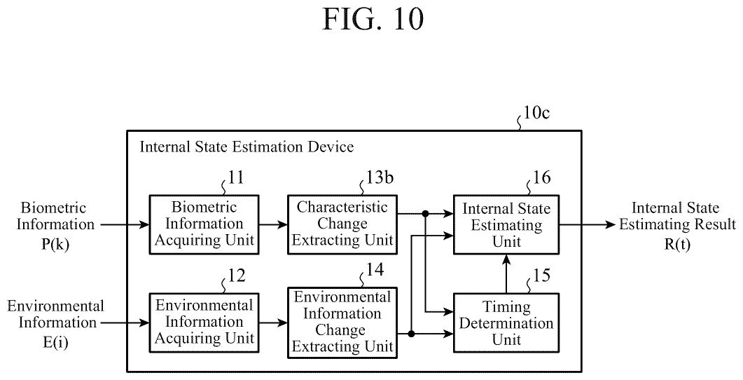

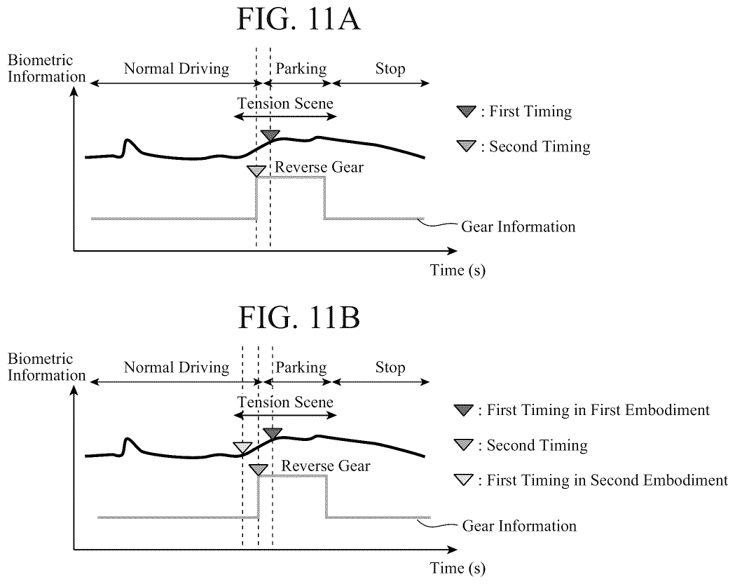

FIG. 10 is a diagram illustrating a configuration example of an internal state estimation device 10c according to the third embodiment. The internal state estimation device 10c according to the third embodiment is different from the internal state estimation device 10 according to the first embodiment illustrated in FIG. 1 in that the characteristic change extracting unit 13 is changed to a characteristic change extracting unit 13b. Since the other configurations of the internal state estimation device 10c according to the third embodiment are the same as those of the internal state estimation device 10 according to the first embodiment illustrated in FIG. 1, the same reference numerals are given and the description thereof will be omitted.

The characteristic change extracting unit 13b acquires biometric information P(k) from the biometric information acquiring unit 11. The characteristic change extracting unit 13b extracts a timing at which a characteristic change in biometric information P(k) starts as a first timing kF on the basis of the biometric information P(k) acquired from the biometric information acquiring unit 11.

For example, the characteristic change extracting unit 13b first extracts a timing at which a characteristic change occurs in the biometric information P(k), similarly to the characteristic change extracting unit 13 in the first embodiment. Next, the characteristic change extracting unit 13b searches for a timing at which a characteristic change starts in the biometric information P(k) by tracing back the timing in the time direction with the extracted timing as a starting point.

For example, in a case where the biometric information P(k) is a heart rate, the characteristic change extracting unit 13b sequentially calculates a differential value at each point of the function indicating the biometric information P(k) while tracing back the timing in the time direction with the timing at which the heart rate exceeds the upper limit threshold as a starting point. Then, the characteristic change extracting unit 13b sets the timing at which the differential value is 0 as the timing at which the characteristic change starts. Alternatively, the characteristic change extracting unit 13b may set, as the timing at which the characteristic change starts, a timing obtained by tracing back the extracted timing in the time direction by a predetermined time (for example, 2 seconds). Then, the characteristic change extracting unit 13b extracts the timing at which the characteristic change starts as the first timing kF.

The characteristic change extracting unit 13b outputs the extracted first timing kF to the timing determination unit 15. In addition, the characteristic change extracting unit 13b sets the biometric information P(k) used for extracting the first timing kF as a characteristic change extracting result F(kF), and outputs the characteristic change extracting result F(kF) to the internal state estimating unit 16.

Next, a specific example and an effect of processing by the characteristic change extracting unit 13b will be described with reference to FIG. 11.

For example, in the internal state estimation device 10 according to the first embodiment, as illustrated in FIG. 11A, the characteristic change extracting unit 13 extracts the timing at which the characteristic change occurs in the biometric information P(k) (for example, the timing at which the heart rate exceeds the upper limit threshold) as the first timing kF on the basis of the biometric information P(k) acquired from the biometric information acquiring unit 11. In this case, depending on the content of the upper limit threshold, the first timing kF may be later than the second timing iZ even though a characteristic change has started to occur in the biometric information P(k).

In that case, the timing determination unit 15 determines that the first timing kF is later than the second timing iZ, and the internal state estimating unit 16 determines that the estimation of the internal state of the occupant is disabled, and the estimation processing of the internal state of the occupant may not be executed. Although there is a possibility that such a case can be avoided depending on the content of the upper limit threshold, it is complicated to reset the upper limit threshold one by one for that purpose, and it may not be realistic from the viewpoint of operation.

In this regard, in the internal state estimation device 10c according to the third embodiment, as illustrated in FIG. 11B, the characteristic change extracting unit 13b extracts a timing at which a characteristic change starts in the biometric information P(k) as the first timing kF instead of the timing at which the characteristic change occurs in the biometric information P(k). As a result, the internal state estimation device 10c according to the third embodiment can more precisely determine the order of the first timing kF and the second timing iZ, and can avoid a problem that the internal state of the occupant is not estimated even though the characteristic change in the biometric information P(k) starts to occur before the change in the environmental information E(i).

Note that, here, an example of configuring the internal state estimation device 10c according to the third embodiment by applying the characteristic change extracting unit 13b to the internal state estimation device 10 according to the first embodiment has been described. However, the internal state estimation device 10c is not limited to this, and may be configured by applying the characteristic change extracting unit 13b to the internal state estimation device 10b according to the second embodiment.

As described above, according to the third embodiment, the characteristic change extracting unit 13b extracts the timing at which the characteristic change in the biometric information P(k) starts as the first timing kF instead of the timing at which the characteristic change occurs in the biometric information P(k). As a result, the internal state estimation device 10c according to the third embodiment can more precisely determine the order of the first timing kF and the second timing iZ in addition to the effects of the first embodiment. As a result, in the internal state estimation device 10c according to the third embodiment, it is possible to avoid a problem in which the internal state of the occupant is not estimated even though the characteristic change in the biometric information P(k) starts to occur before the change in the environmental information E(i).

Note that, in the present disclosure, it is possible to freely combine the respective embodiments, modify any components of the respective embodiments, or omit any components in the respective embodiments.

### INDUSTRIAL APPLICABILITY

The present disclosure can suppress erroneous estimation of an internal state of an occupant of a vehicle, and is suitable for use in an internal state estimation device.

### REFERENCE SIGNS LIST

10: internal state estimation device, 10b: internal state estimation device, 10c: internal state estimation device, 11: biometric information acquiring unit, 12: environmental information acquiring unit, 13: characteristic change extracting unit (first extraction unit), 13b: characteristic change extracting unit, 14: environmental information change extracting unit (second extraction unit), 15: timing determination unit (determination unit), 16: internal state estimating unit (estimation unit), 17: target section setting unit, 21: processing circuit, 22: CPU, 23: memory, E: environmental information, F: characteristic change extracting result, i: input number, iZ: second timing, k: input number, kF: first timing, L: estimation target section, L1: estimation target section, L2: estimation target section, L3: estimation target section, P: biometric information, R: estimation result, RT: determination result, Z environmental information change extracting result

1. An internal state estimation device comprising: processing circuitry
to extract a first timing that is a timing at which a characteristic change occurs in biometric information on a basis of the biometric information acquired in time series from an occupant of a vehicle;
to extract a second timing, which is a timing at which a change occurs in environmental information, on a basis of the environmental information which is information acquired in time series and is information in which a change in the information affects an internal state of the occupant;
to perform determination of an order of the first timing and the second timing; and
to estimate an internal state of the occupant on a basis of at least one of the biometric information and the environmental information, wherein
the processing circuitry determines, depending on a result of the determination whether or not the internal state can be estimated or, on a basis of a result of the determination, estimates the internal state.

2. The internal state estimation device according to claim 1, wherein
the processing circuitry determines it is possible to estimate the internal state in a case where the processing circuitry determines that the first timing is earlier than the second timing, or in a case where the processing circuitry determines that the first timing and the second timing are same.

3. The internal state estimation device according to claim 1, wherein the processing circuitry is further configured
to set an estimation target section having a predetermined time range on a time series in which the biometric information and the environmental information are acquired, wherein
the processing circuitry
extracts the first timing on a basis of a piece of the biometric information included in the estimation target section from the biometric information acquired in time series from the occupant,
the processing circuitry
extracts the second timing on a basis of a piece of the environmental information included in the estimation target section from the environmental information acquired in time series,
the processing circuitry
determines an order of a first timing on a basis of the piece of the biometric information included in the estimation target section and a second timing on a basis of the piece of the environmental information included in the estimation target section, and
the processing circuitry
estimates the internal state of the occupant on a basis of at least one of the piece of the biometric information and the piece of the environmental information included in the estimation target section, and determines, depending on the result of the determination, whether or not the internal state can be estimated or, on a basis of a result of the determination, estimates the internal state.

4. The internal state estimation device according to claim 3, wherein
the processing circuitry moves the estimation target section in a time direction every time a predetermined condition is satisfied.

5. The internal state estimation device according to claim 4, wherein
the predetermined condition is that st pieces of the biometric information (st represents an integer equal to or more than 1) are acquired.

6. The internal state estimation device according to claim 1, wherein
the processing circuitry estimates
the internal state of the occupant by using a database in which at least one of first change information defining a change mode of the biometric information including the characteristic change at the first timing and second change information defining a change mode of the environmental information including the change at the second timing is associated with information indicating the internal state of the occupant.

7. The internal state estimation device according to claim 1, wherein
the biometric information includes at least one of information indicating a heart rate of the occupant, information indicating a pulse rate of the occupant, and information indicating a face image of the occupant.

8. The internal state estimation device according to claim 1, wherein
the environmental information includes at least one of internal information of the vehicle, external information of the vehicle, and control information of the vehicle.

9. The internal state estimation device according to claim 1, wherein
the processing circuitry extracts,
as the first timing, a timing at which a characteristic change in the biometric information starts, instead of the timing at which the characteristic change occurs in the biometric information.

10. An internal state estimation method performed by an internal state estimation device including a processing circuitry, the method comprising:
extracting, by the processing circuitry, a first timing that is a timing at which a characteristic change occurs in biometric information on a basis of the biometric information acquired in time series from an occupant of a vehicle;
extracting, by the processing circuitry, a second timing, which is a timing at which a change occurs in environmental information, on a basis of the environmental information which is information acquired in time series from the vehicle and a surrounding area of the vehicle;
performing, by the processing circuitry, determination of an order of the first timing and the second timing; and
estimating, by the processing circuitry, an internal state of the occupant on a basis of at least one of the biometric information and the environmental information, wherein
the processing circuitry determines, depending on a result of the determination, whether or not the internal state can be estimated or, on a basis of a result of the determination, estimates the internal state.
